(19) United States
(12) Patent Application Publication
(10) Pub. No.: US 2015/0050342 A1
(43) Pub. Date: Feb. 19, 2015

(54) COMPOSITIONS FOR TREATMENT OF SKIN DISORDERS

(71) Applicants: Nicholas Lowe, London (GB); Philippa Lowe, London (GB)

(72) Inventors: Nicholas Lowe, London (GB); Philippa Lowe, London (GB)

(21) Appl. No.: 14/234,311

(22) PCT Filed: Jul. 20, 2012
(86) PCT No.: PCT/GB2012/051744

§ 371 Date: Sep. 19, 2014

(30) Foreign Application Priority Data
Jul. 22, 2011 (GB) 1112657.0
Feb. 17, 2012 (GB) 1202773.6

(51) Int. Cl. A61K 31/60; A61K 31/455; A61K 36/28; A61K 9/00; A61K 31/352; A61K 31/555; A61K 31/355; A61K 45/06; A61K 31/155; A61K 36/815

(52) U.S. Cl. CPC A61K 31/60; A61K 31/155; A61K 31/455; A61K 36/28; A61K 36/815; A61K 31/352; A61K 31/555; A61K 31/355; A61K 45/06; A61K 9/0014
USPC 424/489; 514/161; 424/764; 424/777

(57) ABSTRACT

Compositions are provided comprising: (i) chlorhexidine or a pharmaceutically acceptable salt thereof; (ii) niacinamide or niacin; and (iii) salicylic acid or a pharmaceutically acceptable salt or derivative thereof. The compositions are suitable for use in treating and preventing disorders of the skin. The compositions are also provided in the form of cosmetic products.

This invention relates to novel compositions which are suitable for use in treating and preventing disorders of the skin. In particular, the invention relates to topical compositions which are suitable for use in treating and preventing inflammatory skin disorders. The invention further relates to topical compositions which are suitable for use in treating and preventing skin conditions which are associated with bacterial infections. In particular, the topical compositions of the invention are suitable for use in treating and preventing acne vulgaris. The invention further relates to the use of compositions of the invention as cosmetic products and to methods of preparing the novel topical compositions of the invention.

There is an ongoing need in the art for effective treatments for inflammatory skin disorders, skin disorders associated with bacterial infection, and other types of blemishes. Inflammation of the skin can be caused by a wide variety of factors, including bacterial infection, viral infection, parasitic infection, mycolic infection, allergies, pruritis, vascular disorders, granulomatosis, pigmented dermatosis, and photosensitivity.

Effective treatments for such conditions have a two-fold purpose; to address the underlying cause of the disorder and to treat the physiological symptoms, i.e. the inflammation of the skin. However, a difficulty with many conventional treatments is that active agents which are capable of treating the underlying causes of the disorder, for instance a bacterial infection, are not always well-tolerated at the necessary doses by the subject undergoing treatment. A wide range of side-effects are known, of which irritation, itching and burning sensations are among the most common. Accordingly there remains a need in the art for new treatments which are able to provide relief at least from the symptoms of inflammatory skin disorders, which preferably also address the underlying causes of the disorders, and which are substantially free of the side-effects which can lead to subjects discontinuing their treatment.

A particular class of skin disorders which are addressed by the present invention are those associated with bacterial infection. A wide range of such disorders exist, and are often caused by infection of skin pores (pilosebaceous duct), insect bites and other skin trauma. Examples of skin disorders associated with bacterial infection include acne vulgaris, impetigo, eethyma, folliculitis, carbuncles, sweat gland infections, erysipelas, erythrasma, infected ulcers, infected eczematoid dermatoses, rosacea, and bacterial dermatoses associated with systemic infections. These disorders are usually associated with inflammation of the skin. However, the present invention also addresses the case where bacterial infection is presented in the absence of visible inflammation.

Acne vulgaris (also known as acne, blemishes, spots and breakouts) is a particularly widespread skin disorder which affects people of both sexes between early adolescence and into later years. The term “acne” will be used herein to refer to “acne vulgaris”.

Acne affects a majority of the adolescent population, with some studies suggesting that as many as 95 to 100% of the adolescent population are affected. In adults acne affects between 40 and 50% of the populations and can persist into the 20s, 30s, 40s and even 50s, with 10 to 20% of the population still affected into their 40s. The majority of people who continue to be affected by acne into adulthood are women.

Acne is a disorder of the pilosebaceous duct. Sebaceous glands are found all over the body and are associated with hair follicles and sweat pores. However, the densest populations of sebaceous glands are found on the face, upper chest and the back. The sebaceous glands produce an oily substance known as sebum which usually traverses the pilosebaceous duct to the skin to keep the skin moisturised.

An acne lesion is formed when increased and abnormal cell production of the pilosebaceous ducts leads to blockage of the pilosebaceous duct with keratin cells. This duct blockage is also though to be linked to increased and abnormal production of sebum by the sebaceous glands.

Increased bacterial growth is also associated with the blockage of the pilosebaceous duct, with a variety of strains of proprionobacterium acnes (P. acnes) being the most significant bacteria associated with acne. As sebum and keratin cells continue to accumulate, lesions known as comedones (or more commonly as whiteheads and blackheads) are formed. Inflammation usually develops in the cells surrounding the affected pilosebaceous duct as sebaceous material and P. acnes bacteria leak into the surrounding skin. In particular, P. acnes causes the release of inflammatory compounds known as cytokines into the surrounding skin, leading to the inflammation and scarring that are symptomatic of acne.

There is a significant demand for effective treatments for acne, due to its prevalence and its visibility which often causes affected subjects to feel highly self-conscious about their appearance.

Acne is difficult to treat effectively, and this is reflected in the multiple different treatments which have been proposed. The treatments currently available are aimed at combatting the bacteria associated with acne and the desquamation (unblocking) of the blocked pilosebaceous ducts, as well as reducing the symptoms of acne, such as inflammation of the surrounding skin.

Desquamation of blocked pilosebaceous ducts has been proposed using a variety of different therapeutic agents, including topical retinoids/Vitamin A derivatives (e.g. as gels, lotions and creams) and salicylic acid. Salicylic acid exerts most of its anti-acne effect through desquamation. It reduces the stickiness of stratum corneum cells (corneocytes) of the outer epidermis and comedones, and is therefore effective at removing corneocytes that block the sebaceous duct. However, the use of salicylic acid is limited due to the side effects observed at higher concentrations. These include skin irritation, skin stinging, scaling and dryness.

Reduction of acne bacteria is predominantly with antibacterial agents, usually antibiotics taken systemically (e.g. by mouth) or applied topically to the skin. However, as with many other conditions, the use of antibiotics for the treatment of acne is generally reserved for the most serious cases which are resistant to other forms of treatment due to the significant and growing problem of antibiotic resistance. The use of antibiotics affects all strains of bacteria present in the subject receiving treatment. Consequently, the use of antibiotics can result in the development of resistance in bacterial strains that are not the focus of the treatment. For example Staphylococcus aureus is a bacterium that is commonly found in humans as part of the skin microflora and in most cases is harmless to the host individual. However, the use of antibiotics over many years has led to the emergence of several resistant strains of Staphylococcus aureus which are consequently extremely difficult to treat if they cause illness. In particular, the problem of MRSA (methicillin-resistant Staphylococcus aureus) is well-documented as being responsible for several difficult-to-treat infections in humans. It has been estimated that MRSA was responsible for 94,360 serious infections and associated with 18,650 deaths in the United States in 2005 (Journal of the American Medical Association, 2007, volume 298, page 1763).

In the context of acne treatment, there are a number of antibiotic-resistant strains of P. acnes. The overuse of antibiotics in the treatment of acne further increases the development of antibiotic resistance among bacterial strains associated with acne. In addition, a number of bacterial strains associated with acne (including the P. acnes strains PF282, PF284, PF286, PF287, PF292 and PLF11R) are known already to demonstrate some level of antibiotic resistance. Many subjects are infected by antibiotic resistant P. acnes strains even prior to starting anti-acne therapy, and thus antibiotic treatment for these subjects is ineffective.

Systemic antibiotics also have well-documented side effects that include gastrointestinal upset from Erythromycin and Doxycycline, staining of the skin and teeth from Minocycline, and sunlight sensitivity from Tetracyclines. Other rare, but highly undesirable side effects from systemic antibiotics include severe skin rashes and hair loss.

Topical antibacterial treatment aims to reduce the colonisation of P. acnes and other bacteria, with current antibiotics including Clindamycin and Erythromycin. Again, however, antibiotic-resistant strains of P. acnes reduce the efficacy of these treatments. In addition, the use of these antibiotics in topical acne therapy has been criticised due to the potential for other antibiotic-resistant bacterial strains to develop, leading to a loss of efficacy of these antibiotics for systemic applications. Topical antibiotics have other potential side effects, including stinging, burning, peeling and allergic reactions.

Benzoyl peroxide is widely used in prescription and non-prescription treatment of acne. It reduces growth of P. acnes and other bacteria by generating free radical oxygen species and thus does not lead to bacterial resistance like the antibiotics. Benzoyl peroxide can also be combined with other acne treatments such as topical antibiotics. A problem with bezoyl peroxide is that it can cause skin dryness and skin irritancy, and the concentrations at which it must be used for antibacterial efficacy may be problematic for subjects with sensitive skin. A further potential problem is that the oxygen free radicals generated by benzoyl peroxide, and which kill bacteria, may also lead to collateral skin damage in ways that have not yet been recognised. The chemical nature of benzoyl peroxide also causes bleaching and whitening of clothing and other items with which it may come into contact.

Other treatments with antibacterial activity include photodynamic therapy and blue/red light therapy.

Niacinamide (also known as nicotinamide) is the amide of vitamin B₃ (niacin) and has the formula:

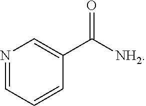

Niacinamide is known to have some anti-inflammatory properties, and its use as an acne therapy has been proposed by Shalita et al. (International Journal of Dermatology, 1995, volume 34, pages 434 to 437). In particular, it was found that topically applied nicotinamide gel (4 to 5% nicotinamide) has comparable efficacy to the antibiotic Clindamycin for the treatment of moderate inflammatory acne vulgaris. However, such an approach is unsatisfactory since nicotinamide has no antibacterial activity and thus affects only the visible signs of inflammation without addressing the underlying physiological and bacterial causes of acne outbreaks.

One problem common to almost all conventional topical treatments for acne and other inflammatory skin disorders is that the treatments cause skin irritation and dryness. This is a particular problem with topical acne treatments that contain benzoyl peroxide, prescription/non-prescription retinoids and salicylic acid. As a result, many subjects become intolerant of their treatment and frequently stop treatment, resulting in a worsening of the condition, more severe skin lesions and scarring.

The present invention provides novel compositions which aim to overcome many or all of the disadvantages of existing treatments for inflammation of the skin. In particular, the present invention provides novel compositions containing a specific combination of components, each of which is present at sub-irritant levels, but which combine synergystically to provide levels efficacy against acne and other inflammatory skin disorders which cannot be obtained using conventional treatments at sub-irritant levels.

In a first aspect, the present invention provides a composition comprising: (i) chlorhexidine or a pharmaceutically acceptable salt thereof; (ii) niacinamide or niacin; and (iii) salicylic acid or a pharmaceutically acceptable salt or derivative thereof.

The composition of the invention preferably comprises chlorhexidine or a pharmaceutically acceptable acid addition salt thereof selected from hydrochloride, hydrobromide, hydroiodide, nitrate, sulfate, bisulfate, phosphate, acid phosphate, acetate, lactate, citrate, tartrate, pantothenate, bitartrate, ascorbate, succinate, maleate, gentisinate, fumarate, gluconate, glucaronate, galactonate, lactobionate, mannonate, saccharate, gulonate, galacturonate, heptagluconate, formate, benzoate, glutamate, methanesulfonate, ethanesulfonate, benzenesulfonate, and p-toluenesulfonate salts.

More preferably, the composition of the invention preferably comprises chlorhexidine or a pharmaceutically acceptable acid addition salt thereof selected from chlorhexidine digluconate, chlorhexidine diacetate, chlorhexidine digalactonate, chlorhexidine dilactobionate, chlorhexidine dimannonate, chlorhexidine digulonate, chlorhexidine digalacturonate, and chlorhexidine diheptagluconate. Still more preferably, the composition of the invention comprises chlorhexidine diacetate or chlorhexidine digluconate, and most preferably chlorhexidine digluconate.

Chlorhexidine digluconate is known in the art for use in dental applications, antiseptic cleaning compositions and as a preservative in cosmetic formulations. However, its use for the treatment and prevention of inflammatory skin disorders has found little attention. It is thought that this is because it shows little activity at low concentrations and becomes irritating to skin and mucosal membranes at higher concentrations.

It has now surprisingly been found that chlorhexidine and pharmaceutically acceptable salts thereof (particularly chlorhexidine digluconate) are highly effective for the treatment and prevention of inflammatory skin disorders when used in the compositions according to the present invention. Without being bound by any particular mechanism of action, it is believed that the antibacterial activity of chlorhexidine and pharmaceutically acceptable salts thereof is enhanced in the presence of niacinamide/niacin and salicylic acid or salts/derivatives thereof. Thus, it may be used effectively at concentrations which provide effective activity against inflammatory skin disorders, but below the level at which skin and mucosal toxicity is observed.

As a further benefit, it has been found that the antibacterial activity of chlorhexidine and pharmaceutically acceptable salts thereof in the compositions of the invention is retained for several hours after application, providing continued inhibition of bacterial growth.

The composition of the invention preferably comprises niacinamide.

The composition of the invention preferably comprises salicylic acid, O-acetylsalicylic acid (also known as 2-acetoxybenzoic acid) or a pharmaceutically acceptable salt of salicylic acid selected from sodium salicylate, magnesium salicylate, and choline salicylate. Most preferably, the composition of the invention comprises salicylic acid.

The composition of the invention preferably comprises: (i) chlorhexidine or a pharmaceutically acceptable salt thereof in an amount of from 5 to 30 parts by weight; (ii) niacinamide or niacin in an amount of from 50 to 500 parts by weight; and (iii) and salicylic acid or a pharmaceutically acceptable salt or derivative thereof in an amount of from 20 to 500 parts by weight.

References herein to parts by weight of chlorhexidine or a pharmaceutically acceptable salt thereof refer to the equivalent weight of chlorhexidine digluconate.

References herein to parts by weight of niacinamide or niacin refer to the equivalent weight of niacinamide.

References herein to parts by weight of salicylic acid or a pharmaceutically acceptable salt or derivative thereof refer to the equivalent weight of salicylic acid.

More preferably, the composition of the invention comprises chlorhexidine or a pharmaceutically acceptable salt thereof in an amount of at least 8 parts by weight, and more preferably at least 10 parts by weight. More preferably, the composition of the invention comprises chlorhexidine or a pharmaceutically acceptable salt thereof in an amount of 28 parts by weight or less, more preferably 25 parts by weight or less, still more preferably 23 parts by weight or less, and most preferably 20 parts by weight or less.

More preferably, the composition of the invention comprises niacinamide or niacin in an amount of at least 80 parts by weight, more preferably at least 100 parts by weight, and most preferably at least 150 parts by weight. More preferably, the composition of the invention comprises niacinamide or niacin in an amount of 450 parts by weight or less, more preferably 400 parts by weight or less, and most preferably 350 parts by weight or less.

More preferably, the composition of the invention comprises salicylic acid or a pharmaceutically acceptable salt or derivative thereof in an amount of at least 30 parts by weight, preferably at least 40 parts by weight, more preferably at least 50 parts by weight, yet more preferably at least 80 parts by weight, still more preferably at least 100 parts by weight, and most preferably at least 150 parts by weight. More preferably, the composition of the invention comprises salicylic acid or a pharmaceutically acceptable salt or derivative thereof in an amount of 450 parts by weight or less, more preferably 400 parts by weight or less, and most preferably 350 parts by weight or less.

In a preferred embodiment, the composition of the invention comprises: (i) chlorhexidine digluconate in an amount of from 5 to 30 parts by weight; (ii) niacinamide in an amount of from 50 to 500 parts by weight; and (iii) and salicylic acid in an amount of from 20 to 500 parts by weight.

More preferably, the composition of the invention comprises chlorhexidine digluconate in an amount of at least 8 parts by weight, and more preferably at least 10 parts by weight. More preferably, the composition of the invention comprises chlorhexidine digluconate in an amount of 28 parts by weight or less, more preferably 25 parts by weight or less, still more preferably 23 parts by weight or less, and most preferably 20 parts by weight or less.

More preferably, the composition of the invention comprises niacinamide in an amount of at least 80 parts by weight, more preferably at least 100 parts by weight, and most preferably at least 150 parts by weight. More preferably, the composition of the invention comprises niacinamide in an amount of 450 parts by weight or less, more preferably 400 parts by weight or less, and most preferably 350 parts by weight or less.

More preferably, the composition of the invention comprises salicylic acid in an amount of at least 30 parts by weight, preferably at least 40 parts by weight, more preferably at least 50 parts by weight, yet more preferably at least 80 parts by weight, still more preferably at least 100 parts by weight, and most preferably at least 150 parts by weight. More preferably, the composition of the invention comprises salicylic acid in an amount of 450 parts by weight or less, more preferably 400 parts by weight or less, and most preferably 350 parts by weight or less.

The salicylic acid (or pharmaceutically acceptable salt/derivative thereof) component of the composition is preferably encapsulated. Encapsulation is used in the present invention in order to regulate the rate of delivery of the active agent to the skin. In this way, a sufficient concentration of salicylic acid (or pharmaceutically acceptable salt/derivative thereof) is maintained to obtain the required desquamation properties, but skin irritation due to excessive concentration is substantially avoided.

The delivery of active agents in encapsulated form is known to persons skilled in the art, with many different types of encapsulation available which allow for the regulated delivery of the active agents over a period ranging from a few minutes to several hours. A common technique, compatible with the present invention, involves the encapsulation of salicylic acid particles in phospholipid vesicles. The encapsulation of salicylic based on non-phospholipid vesicles is also known in the art. Typically, the encapsulated salicylic acid has a number average particle size of less than 1.0 μm. In accordance with the present invention, the salicylic acid is preferably released from the encapsulated form over a period of at least 30 minutes, more preferably at least one hour, and most preferably at least two hours.

In a preferred embodiment of the invention, the composition of the invention further comprises eucalyptol. Eucalyptol (also known as cineole) forms up to 90% of eucalyptus globulus extract, and can also be obtained from number of other plant sources. It has the formula:

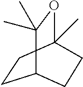

Again, without being limited by any particular mechanism, it is believed that the effect of eucalyptol in the compositions of the invention is to supplement the antibacterial effect of chlorhexidine digluconate. It has been found that chlorhexidine gluconate and eucalyptol combine to provide high levels of antibacterial activity. However, it has been found that the level of skin irritation associated with the combined use of these substances is substantially reduced compared to that which would be observed when using either component alone to achieve a similar level of antibacterial activity. Thus, while the combination of chlorhexidine gluconate and eucalyptol is constructive in terms of antibacterial activity, these two agents appear to follow different physiological pathways as skin irritants, and thus their combined use does not entail an increase in skin irritation.

The composition of the invention preferably comprises eucalyptol in an amount of from 30 to 500 parts by weight. In further preferred embodiments, the amount of eucalyptol is at least 50 parts by weight, still more preferably at least 70 parts by weight, and most preferably at least 90 parts by weight. In further preferred embodiments, the amount of eucalyptol is 450 parts by weight or less, still more preferably 400 parts by weight or less, and most preferably 350 parts by weight or less.

In a further preferred embodiment of the invention, eucalyptol is provided in the form of eucalyptus globulus extract (also known as eucalyptus extract and eucalyptus oil). Eucalyptus globulus extract is an oil distilled from the leaves of Eucalyptus globulus. The eucalyptol content of eucalyptus globulus extract can very depending on the source of the extract. In accordance with the present invention, the eucalyptus globulus extract preferably has a eucalyptol content of at least 60 wt %, and more preferably at least 70 wt %.

Where eucalyptol is provided in the form of eucalyptus globulus extract, the amount of eucalyptus globulus extract in the composition of the invention is preferably in the range of from 50 to 500 parts by weight. In further preferred embodiments, the amount of eucalyptus globulus extract is at least 80 parts by weight, more preferably at least 100 parts by weight, still more preferably at least 150 parts by weight, and most preferably at least 200 parts by weight. In further preferred embodiments, the amount of eucalyptus globulus extract is 450 parts by weight or less, more preferably 400 parts by weight or less, and most preferably 350 parts by weight or less.

In a further preferred embodiment, the composition of the invention further comprises zinc-PCA. Zinc-PCA is the zinc salt of L-pyroglutamic acid (also known as zinc pyroglutamate, the zinc salt of L-pyrrolidone carboxylic acid, and zincidone) and is believed to function in the compositions of the invention to reduce the secretion of sebum. As noted above, excessive and abnormal production of sebum is an important contributory factor in the development of acne lesions as well as in other inflammatory skin disorders. By reducing sebum production, zincidone further enhances the activity of chlorhexidine digluconate, salicylic acid and niacinamide in the compositions of the invention.

The zinc-PCA content of the composition of the invention is preferably in the range of from 10 to 300 parts by weight. In further preferred embodiments, the amount of zinc-PCA is at least 20 parts by weight, more preferably at least 40 parts by weight, still more preferably at least 60 parts by weight, still more preferably at least 80 parts by weight, and most preferably at least 100 parts by weight. In further preferred embodiments, the amount of Zinc-PCA is 280 parts by weight or less, more preferably 260 parts by weight or less, still more preferably 240 parts by weight or less, still more preferably 220 parts by weight or less, and most preferably 200 parts by weight or less.

In addition to the active ingredients mentioned above, the efficacy of the composition of the invention may be further enhanced by the inclusion of one or more antioxidants. Antioxidants are known to have anti-inflammatory properties which make them suitable for treating a number of different inflammatory skin disorders. The reduction of inflammation is an important characteristic of treatments for many skin disorders, particularly acne, since it is inflammation which can lead to scarring of the skin which remains following remission of the disorder.

One preferred antioxidant for use in the composition of the invention is tocopherol. The anti-inflammatory properties of topical tocopherol are known, however there is not believed to be any documented use of tocopherol in anti-acne compositions. Tocopherol is preferably present in the composition of the invention in an amount of from 50 to 500 parts by weight.

Another preferred antioxidant for use in the composition of the invention is Goji extract (also known as Lyceum barburum fruit extract), a natural antioxidant obtained from the fruit of Lyceum barburum goji berries. Goji extract is known to have efficacy against skin inflammation by preventing the skin from lipid peroxidation. Goji extract is preferably present in the composition of the invention in an amount of from 50 to 500 parts by weight.

A further active ingredient which may be included in the compositions of the invention for its anti-inflammatory properties is chamomile extract (also known as chamomile essential oil). Chamomile extract is obtained from the Anthemis nobilis plant or the Maticaria recutita plant and its anti-inflammatory properties are believed to be attributable, at least in part, to the active components bisabolol and azulune. Chamomile extract has found application as an anti-inflammatory in the treatment of eczema and skin inflammation from radiation therapy. However, there are believed to be no reports of the use of chamomile extract for the treatment of acne-related inflammation. Chamomile extract is preferably present in the composition of the invention in an amount of from 50 to 300 parts by weight.

The amount of chlorhexidine or a pharmaceutically acceptable salt thereof in the compositions of the invention is preferably in the range of from 0.05 to 0.3 wt %. In further preferred embodiments, the amount of chlorhexidine or a pharmaceutically acceptable salt thereof is at least 0.08 wt %, and still more preferably at least 0.1 wt %. In further preferred embodiments, the amount of chlorhexidine or a pharmaceutically acceptable salt thereof is 0.28 wt % or less, more preferably 0.25 wt % or less, still more preferably 0.23 wt % or less, and most preferably 0.2 wt % or less.

The amount of niacinamide or niacin in the composition of the invention is preferably in the range of from 0.5 to 5 wt %. In further preferred embodiments, the amount of niacinamide or niacin is at least 0.8 wt %, still more preferably at least 1 wt %, and most preferably at least 1.5 wt %. In further preferred embodiments, the amount of niacinamide or niacin is 4.5 wt % or less, still more preferably 4.0 wt % or less, and most preferably 3.5 wt % or less. Without being bound by any particular theory, it is believed that the use of niacinamide or niacin in these amounts provides effective anti-inflammatory action, whilst also enhancing the activity of chlorhexidine and salicylic acid (and/or the pharmaceutically acceptable salts thereof).

The amount of salicylic acid or a pharmaceutically acceptable salt or derivative thereof in the composition of the invention is preferably in the range of from 0.2 to 5 wt %. In further preferred embodiments, the amount of salicylic acid or a pharmaceutically acceptable salt or derivative thereof is at least 0.3 wt %, preferably at least 0.4 wt %, more preferably 0.5 wt %, yet more preferably at least 0.8 wt %, still more preferably at least 1 wt %, and most preferably at least 1.5 wt %. In further preferred embodiments, the amount of salicylic acid or a pharmaceutically acceptable salt or derivative thereof is 4.5 wt % or less, still more preferably 4.0 wt % or less, and most preferably 3.5 wt % or less.

References herein to wt % of chlorhexidine or a pharmaceutically acceptable salt thereof refer to the equivalent weight of chlorhexidine digluconate.

References herein to wt % of niacinamide or niacin refer to the equivalent weight of niacinamide.

References herein to wt % of salicylic acid or a pharmaceutically acceptable salt or derivative thereof refer to the equivalent weight of salicylic acid.

As used herein, amounts expressed in terms of percent by weight (wt %) refer to the percentage by weight of the entire composition, including any excipients/additives which may be present.

In a preferred embodiment, the compositions of the invention comprise chlorhexidine digluconate in an amount in the range of from 0.05 to 0.3 wt %. In more preferred embodiments, the amount of chlorhexidine digluconate is at least 0.08 wt %, and still more preferably at least 0.1 wt %. In more preferred embodiments, the amount of chlorhexidine digluconate is 0.28 wt % or less, more preferably 0.25 wt % or less, still more preferably 0.23 wt % or less, and most preferably 0.2 wt % or less.

In a further preferred embodiment, the compositions of the invention comprise niacinamide in an amount in the range of from 0.5 to 5 wt %. In more preferred embodiments, the amount of niacinamide is at least 0.8 wt %, still more preferably at least 1 wt %, and most preferably at least 1.5 wt %. In more preferred embodiments, the amount of niacinamide is 4.5 wt % or less, still more preferably 4.0 wt % or less, and most preferably 3.5 wt % or less. Without being bound by any particular theory, it is believed that the use of niacinamide in these amounts provides effective anti-inflammatory action, whilst also enhancing the activity of chlorhexidine digluconate and salicylic acid.

In a further preferred embodiment, the compositions of the invention comprise salicylic acid in an amount in the range of from 0.2 to 5 wt %. In more preferred embodiments, the amount of salicylic acid is at least 0.3 wt %, preferably at least 0.4 wt %, more preferably at least 0.5 wt % yet more preferably at least 0.8 wt %, still more preferably at least 1 wt %, and most preferably at least 1.5 wt %. In more preferred embodiments, the amount of salicylic acid is 4.5 wt % or less, still more preferably 4.0 wt % or less, and most preferably 3.5 wt % or less.

In general, higher amounts of salicylic acid (e.g. from 3.5 to 5 wt %) can be used when the salicylic acid is encapsulated, whereas lower amounts (e.g. 3.5 wt % or less) are most preferred where the salicylic acid is unencapsulated. References to wt % of encapsulated salicylic acid as used herein refer to the salicylic acid content of the encapsulated salicylic acid.

In preferred embodiments, the composition of the invention comprises eucalyptol in an amount in the range of from 0.3 to 5 wt %. In further preferred embodiments, the amount of eucalyptol is at least 0.5 wt %, still more preferably at least 0.7 wt %, and most preferably at least 0.9 wt %. In further preferred embodiments, the amount of eucalyptol is 4.5 wt % or less, still more preferably 4.0 wt % or less, and most preferably 3.5 wt % or less. The use of eucalyptol in these ranges avoids skin irritation and the pungent smell associated with eucalyptol, which some people find unpleasant.

Alternatively, as noted above, eucalyptol may be provided in the form of eucalyptus globulus extract. Where eucalyptol is provided in the form of eucalyptus globulus extract, the amount of eucalyptus globulus extract in the composition of the invention is preferably in the range of from 0.5 to 5 wt %. In further preferred embodiments, the amount of eucalyptus globulus extract is at least 0.8 wt %, still more preferably at least 1 wt %, and still more preferably at least 1.5 wt %, and most preferably at least 2.0 wt %. In further preferred embodiments, the amount of eucalyptus globulus extract is 4.5 wt % or less, still more preferably 4.0 wt % or less, and most preferably 3.5 wt % or less. As above, the use of eucalyptus globulus extract in these ranges avoids skin irritation and the pungent smell associated with eucalyptus globulus extract, which some people find unpleasant.

In another preferred embodiment, the composition of the invention comprises zinc-PCA in an amount in the range of from 0.1 to 3 wt %. In further preferred embodiments, the amount of zinc-PCA is at least 0.2 wt % more preferably at least 0.4 wt %, still more preferably at least 0.6 wt %, still more preferably at least 0.8 wt % and most preferably at least 1 wt %. In further preferred embodiments, the amount of zinc-PCA is 2.8 wt % or less, more preferably 2.6 wt % or less, still more preferably 2.4 wt % or less, still more preferably 2.2 wt % or less and most preferably 2.0 wt % or less.

In another preferred embodiment, the composition of the invention comprises tocopherol in an amount of from 0.5 to 5 wt %.

In another preferred embodiment, the composition of the invention comprises Goji extract in an amount of from 0.5 to 5 wt %.

In another preferred embodiment, the composition of the invention comprises chamomile extract in an amount of from 0.5 to 3 wt %.

In some embodiments, the composition of the invention may be provided as a concentrate which is suitable for formulation into a pharmaceutical composition.

In further embodiments, the composition of the invention is in the form of a pharmaceutical composition which is suitable for topical administration.

In further embodiments, the composition of the invention is in the form of a cosmetic composition which is suitable for topical administration.

In preferred embodiments, the compositions of the invention may further comprise one or more excipients suitable for topical administration.

As defined herein, the term excipient refers to an inactive substance which is used as a carrier vehicle for the active compounds discussed above. Suitable excipients are well-known to the person of skill in the art, and include oils, waxes, solvents, emollients, stabilisers, thickeners, emulsifiers, co-emulsifiers, preservatives, moisturisers, sebum absorbers, skin conditioners, buffers, pH adjusters, chelating agents, bulking agents, surfactants, film formers, exfoliants, astringents, colouring agents/dyes, fragrances, foamers and antifoaming agents. These substances may be used independently of each other in a manner which is well-known by those of skill in the art. The following paragraphs provide details of excipients which are merely exemplary of those which may be used in accordance with the invention and are not intended to be limiting.

Emollients may be used in the compositions of the invention to prevent or relieve dryness. Suitable emollients include triglyceride esters (such as caprylic triglyceride and capric triglyceride), silicone oils, alkyl esters, fatty acid esters, fatty alcohols, paraffins, isoparaffins (such as isohexadecane), lanolin and derivatives, polyethers, polyols and polyol esters, beeswax and vegetable waxes.

Suitable stabilisers may be non-ionic, cationic, anionic or amphiphilic. Examples include sodium stearoyl glutamate, hydroxyethylacrylate/sodium acryloyldimethyltaurate copolymer, sorbitan isostearate, Polysorbate 60, xanthan gum and carbomer.

Thickeners suitable for use in the compositions of the invention include cetearyl alcohol and beeswax.

Emulsifiers are used to maintain uniform blending of the compositions of the invention. Suitable emulsifiers may be non-ionic, cationic or anionic. Anionic emulsifiers include fatty acid soaps (e.g. sodium stearate and ammonium stearate), polyol fatty acid esters containing fatty acid soaps (e.g. sodium stearoyl glutamate), fatty acids (such as stearic acid) and fatty sulfate salts. Suitable cationic emulsifiers include ammonium and pyridinium salts. Suitable non-ionic emulsifiers include polyoxyethylene fatty acid esters (such as PEG-20 stearate and PEG-100 stearate), polyol fatty acid esters (such as glyceryl stearate) and fatty alcohols (such as stearyl and cetyl alcohols, or a mixture thereof known as cetearyl alcohol).

Suitable preservatives include, propylparaben, methylparaben, phenoxyethanol, phenethyl alcohol, caprylyl glycol, benzoic acid, dehydroacetic acid, and ethylhexylglycerin.

Suitable moisturisers/humectants include glycerol, propylene glycol, glycereth-18, and glycereth-18 ethylhexanoate

Suitable sebum absorbers include aluminium starch octenylsuccinate, silica silylates and clays.

Suitable skin conditioners include allantoin, myristamidopropyl PG-dimonium chloride phosphate, and PEG-7 glyceryl cocoate

Suitable chelating agents include disodium EDTA and tetrasodium EDTA.

Suitable pH adjusters include sodium hydroxide, ammonium hydroxide and citric acid. The pH of the compositions of the invention is preferably in the range of from 3 to 8, more preferably 4 to 7 and most preferably 5 to 6.

Suitable bulking agents include kaolin and other clays, silica, starch and cellulose.

Surfactants may be included as cleansing agents, foamers, solubilising agents, and homogenisers. Examples include coco-glucoside and decyl glucoside.

Exfoliants may be used to assist the removal of outer skin cells (corneocytes). Examples include polyethylene particles and Jojoba esters.

Suitable solvents include water and alcohols. Alcohols, such as ethanol and denatured alcohol, also have astringent properties.

The composition of the invention may take a number of different forms. In preferred embodiments of the invention, the composition of the invention is formulated as a cream (e.g. a blemish cream), ointment, scrub, gel, cleanser, toner, gel mask, or clay mask.

In a further preferred embodiment, the present invention provides compositions as defined in Tables 1 to 5 below.

In accordance with a further aspect of the invention, there are provided textile cloths, wipes, pads and face masks impregnated with the composition of the invention. The skilled person will recognise that certain formulations, such as certain clays, may be unsuitable for impregnating textiles, and can readily identify and prepare compositions suitable for this purpose.

In accordance with a further aspect of the invention, there is also provided a composition as defined above for use in therapy.

In accordance with a further aspect of the invention, there is also provided a composition as defined above for use in treating and/or preventing skin disorders associated with inflammation of the skin.

Preferably the skin disorders are selected from bacterial dermatoses, allergic dermatoses, allergic dermatoses, pruritic dermatoses, vascular dermatoses, viral dermatoses, mycolic skin infections, granulomatous skin infections, parasitic skin infections, pigmented dermatoses, rosacea and photosensitive dermatoses.

In accordance with a further aspect of the invention, there is also provided a composition as defined above for use in treating and/or preventing bacterial dermatoses.

More preferably the bacterial dermatoses are selected from acne vulgaris, impetigo, eethyma, folliculitis, carbuncles, sweat gland infections, erysipelas, erythrasma, infected ulcers, infected eczematoid dermatoses, rosacea as well as bacterial dermatoses associated with systemic infections.

Most preferably, the present invention provides a composition as defined above for use in treating and/or preventing acne vulgaris. Preferably, the composition is provided for use in treating and/or preventing acne vulgaris associated with Propionibacterium acnes infection.

In a further aspect, the present invention provides the use of a composition as defined above for treating and/or preventing an inflammatory skin disorder. Preferably, the inflammatory skin disorder is as defined above.

In a further aspect, the present invention provides the use of a composition as defined above for treating and/or preventing bacterial dermatoses. Preferably, the bacterial dermatoses are as defined above.

The compositions of the invention may also be used for cosmetic purposes, for instance as a skin cleanser or a skin toner. Thus, in a further aspect, the present invention provides the use of a composition as defined above as a cosmetic. Preferably, the compositions for use as a cosmetic are formulated with one or more excipients suitable for topical administration as described above.

The compositions of the invention have antibacterial activity and may also therefore be used for hygienic purposes, for instance for sanitising the hands. Thus, in a further aspect, the present invention provides the use of a composition as defined above as a topical antibacterial agent.

The present invention will now be illustrated by reference to the following Examples.

### EXAMPLES

### Example 1

### Day and Night Cream

A composition according to the present invention and formulated for use as a cream for application during the day or at night is shown in Table 1.

TABLE 1

| Component | Function | Amount (wt %) |
|---|---|---|
| Chlorhexidine gluconate | Active | 0.2 to 0.3 |
| Niacinamide | Active | 2 to 3 |
| Encapsulated salicylic acid | Active | 3 to 5 |
| Eucalyptus globulus extract | Active | 0.5 to 2 |
| Goji extract | Active | 1 to 3 |
| Zinc-PCA | Active | 0.1 to 0.5 |
| Tocopherol | Active | 1 to 3 |
| Chamomile extract | Active | 0.5 to 2 |
| Isohexadecane | Emollient | Balance |
| Sodium stearoyl glutamate | Stabiliser/Co-emulsifier | 1 |
| Cetearyl alcohol | Thickener/Co-emulsifier | 1 |
| Cetearyl glucoside | Emulsifier | 1 |
| Propyl paraben | Preservative | 0.1 |
| Methyl paraben | Preservative | 0.1 |
| Phenoxyethanol | Preservative | 0.1 |
| Glycerol | Humectant/Moisturiser | 5 to 10 |
| Hydroxyethylacrylate/sodium | Stabiliser/Co-emulsifier | 1 |
| acryloyl dimethyltaurate | | |
| copolymer | | |
| Sorbitan isostearate | Stabiliser/Co-emulsifier | 1 |
| Polysorbate 60 | Stabiliser/Co-emulsifier | 1 |
| Aluminium starch | Sebum absorber | 3 to 5 |
| octenylsuccinate | | |
| Allantoin | Skin conditioner | 0.1 |
| Disodium EDTA | Chelating agent | 0.05 |
| Sodium hydroxide | pH adjuster | 0.1 |

### Example 2

### Mask

A composition according to the present invention and formulated for use as a deep pore mask is shown in Table 2.

TABLE 2

| Component | Function | Amount (wt %) |
|---|---|---|
| Chlorhexidine gluconate | Active | 0.2 to 0.3 |
| Niacinamide | Active | 2 to 3 |
| Encapsulated salicylic acid | Active | 3 to 5 |
| Eucalyptus globulus extract | Active | 0.5 to 2 |
| Goji extract | Active | 1 to 3 |
| Zinc-PCA | Active | 0.1 to 0.5 |
| Tocopherol | Active | 1 to 3 |
| Chamomile extract | Active | 0.5 to 2 |
| Cetearyl alcohol | Emulsifier | 1 |
| PEG-20 stearate | Emulsifier | 1 |
| Kaolin | Bulking agent | 3 to 5 |
| Coco-glucoside | Surfactant | 1 |
| Beeswax | Thickener | 3 to 5 |
| Xanthan gum | Stabiliser | 1 |
| Phenethyl alcohol | Preservative | 0.1 |
| Caprylyl glycol | Preservative | 0.1 |
| Sodium hydroxide | pH adjuster | 0.1 |

### Example 3

### Scrub

A composition according to the present invention and formulated for use as a deep pore scrub is shown in Table 3.

TABLE 3

| Component | Function | Amount (wt %) |
|---|---|---|
| Chlorhexidine gluconate | Active | 0.2 to 0.3 |
| Niacinamide | Active | 2 to 3 |
| Encapsulated salicylic acid | Active | 3 to 5 |
| Eucalyptus globulus extract | Active | 0.5 to 2 |
| Goji extract | Active | 1 to 3 |
| Stearic acid | Emulsifier | 1 |
| Glyceryl stearate | Emulsifier | 1 |
| PEG-100 stearate | Emulsifier | 1 |
| Carbomer | Stabiliser/Thickener | 0.5 |
| Capric/caprylic triglycerides | Emollient | Balance |
| Cetyl alcohol | Co-emulsifier/thickener | 0.5 |
| Coco-glucoside | Surfactant | 0.5 |
| Glycerol | Humectant/Moisturiser | 5 to 10 |
| Polyethylene | Exfoliant | 1 to 3 |
| Jojoba extract | Exfoliant | 1 to 3 |
| Phenoxyethanol | Preservative | 0.1 |
| Benzoic acid | Preservative | 0.1 |
| Dehydroacetic acid | Preservative | 0.1 |
| Ethylhexylglycerin | Preservative | 0.1 |
| Sodium hydroxide | pH adjuster | 0.1 |

### Example 4

### Spot Gel

A pharmaceutical according to the present invention and formulated for use as a spot gel is shown in Table 4.

TABLE 4

| Component | Function | Amount (wt %) |
|---|---|---|
| Chlorhexidine gluconate | Active | 0.2 to 0.3 |
| Niacinamide | Active | 2 to 3 |
| Salicylic acid | Active | 2 to 4 |
| Eucalyptus globulus extract | Active | 0.5 to 2 |
| Goji extract | Active | 1 to 3 |
| Chamomile extract | Active | 0.5 to 2 |
| Allantoin | Skin conditioner | 0.1 |
| Denatured alcohol | Astringent | 5 |
| Hydroxyethyl cellulose | Thickener | 5 |
| Propylene glycol | Humectant/Solubiliser | 5 |
| Water | | Balance |

### Example 5

### Cleanser and Toner

A composition according to the present invention and formulated for use as a cleanser and toner is shown in Table 5.

TABLE 5

| Component | Function | Amount (wt %) |
|---|---|---|
| Chlorhexidine gluconate | Active | 0.2 to 0.3 |
| Niacinamide | Active | 2 to 3 |
| Salicylic acid | Active | 2 to 4 |
| Eucalyptus globulus extract | Active | 0.5 to 2 |
| Goji extract | Active | 1 to 3 |
| Zinc-PCA | Active | 0.1 to 0.5 |
| Glycereth 18 ethylhexanoate | Humectant/Moisturiser | 2.5 |
| Glycereth 18 | Humectant/Moisturiser | 2.5 |
| Myristamidopropyl PG-dimonium | Skin conditioner | 2.5 |
| chloride phosphate | | |
| PEG-7 glyceryl cocoate | Skin conditioner | 1 |
| Allantoin | Skin conditioner | 0.1 |
| Decyl glucoside | Surfactant | 2 |
| Disodium EDTA | Chelating agent | 0.05 |
| Phenoxyethanol | Preservative | 0.1 |
| Benzoic acid | Preservative | 0.1 |
| Dehydroacetic acid | Preservative | 0.1 |
| Ethylhexylglycerin | Preservative | 0.1 |
| Sodium hydroxide | pH adjuster | 0.1 |
| Water | Solvent | Balance |

### Example 6

### Skin Irritancy Test

Fifty-two subjects were studied with a standard repeat application product skin test using a composition as defined in Table 1 under occlusion and semi-occlusion. No evidence of skin irritancy was observed during these studies.

### Example 7

### Allergy Test

A composition defined in Table 1 was reapplied to the backs of the same subjects as in Example 6 after a period of 14 days had elapsed from the skin irritancy testing of Example 6. No allergic skin reactions were observed.

### Example 8

### In Vitro Propionibacterium Acnes Inhibitory Test—General Procedure

Compositions as defined in Tables 1 to 5 were analysed using the in vitro Propionibacterium spp inhibition test developed by the Leeds Skin Centre for Applied Research. This antibacterial assay is considered by acne experts to be highly predictive of anti-acne activity of a product and uses serial two-fold dilution to determine the Minimum Inhibitory Concentrations (MIC) of the compositions of the invention in the range of 10% to 0.005% (w/v). Twenty different strains of Propionibacterium acnes are considered, seven of which were selected because they are resistant to antibiotics routinely used for acne therapy.

Testing was carried out at ambient temperature in a UKAS accredited testing laboratory No. 4082.

Propinibacteria isolates were routinely maintained on Reinforced Clostridial Agar with 6 μg/mL furazolidone, anaerobically at 34° C. Isolates were grown to late exponential phase of growth by inoculating Tryptone Yeast Extract Glucose broth with 3 to 4 individual colonies and incubating for 3 days anaerobically without shaking at 34° C.

The propionibacteria strains tested, together with their antibiotic resistance profile are shown in Table 6.

TABLE 6

| | Organism ID | |
|---|---|---|
| | (propionibacterium | |
| | spp.) | Antimicrobial Resistance Profile |
| 1 | AT4 | Fully susceptible |
| 2 | P506 | Fully susceptible |
| 3 | P37 | Fully susceptible |
| 4 | NCTC 737 | Fully susceptible |
| 5 | PF284 | Tetracycline resistant |
| 6 | PF286 | Tetracycline resistant |
| 7 | AT1 | Fully susceptible |
| 8 | 101941B | Fully susceptible |
| 9 | 101850A | Fully susceptible |
| 10 | 101842C | Fully susceptible |
| 11 | PV63S | Fully susceptible |
| 12 | PF211 | Fully susceptible |
| 13 | Cn6966 | Fully susceptible |
| 14 | 102054I | Fully susceptible |
| 15 | PF287 | Erythromycin resistant, Clindamycin resistant |
| 16 | PrZ103640E | Erythromycin resistant |
| 17 | PrZ103602 | Fully susceptible |
| 18 | PF282 | Septrin resistant (sulphamethoxazole |
| | | trimethoprim) |
| 19 | Pf292 | Erythromycin resistant, Clindamycin resistant |
| 20 | PLF11R | Erythromycin resistant, Tetracycline resistant |

### Preparation of Agar Dilution Plates

Test products were prepared in triplicate as 10× concentration stock solutions. The highest test concentration for each test item was 512 mg/L (stock at 5120 mg/L). Serial doubling dilutions of each test item were prepared using water as a solvent. For each test and control item, 2 mL of 10× stock was mixed with 18 mL Reinforced Clostridial Agar (RCA) at 1.1× concentration in triplicate. Control RCA plates containing no test items were prepared at the start and end of each dilution series.

### Inoculation of Agar Plates

Propionibacterial inocula were prepared to a density of 1×10⁵ cfu/μL by adjusting the optical density (600 nm) of the cultures to 5.0. An AQS A400 Multipoint Inoculator was used to inoculate each plate with a 10 μL spot of inoculum. Inoculated plates were incubated anaerobically for 7 days at 34° C. Following incubation, the plates were examined against a dark background. Growth was evaluated on a scale of 0 to 3 where 0 corresponds to no detected growth and 3 corresponds to growth equivalent to the control plate. Each triplicate concentration (including the control) could therefore achieve a total maximum score of 9. Total scores between 0 and 3 are classed as no growth detected.

### Example 9

### Inhibition of Propionibacterium Acnes by Compositions of the Invention

The inhibition of Propionibacterium acnes by a composition of the invention in the form of a cream as defined in Example 1 was examined according to the general procedure identified in Example 8. Results of inhibition testing are shown in Table 7 and calculated MIC values are shown in Table 8.

TABLE 7

| | Dilution % | 10 | 5.0 | 2.5 | 1.25 | 0.63 | 0.31 | 0.16 |
|---|---|---|---|---|---|---|---|---|
| 1 | AT4 | 0 | 0 | 0 | 0 | 0 | 0 | 5 |
| 2 | P506 | 0 | 0 | 0 | 0 | 0 | 0 | 9 |
| 3 | P37 | 0 | 0 | 0 | 0 | 0 | 0 | 0 |
| 4 | NCTC 737 | 0 | 0 | 0 | 0 | 0 | 0 | 9 |
| 5 | PF284¹ | 0 | 0 | 0 | 0 | 0 | 0 | 9 |
| 6 | PF286 | 0 | 0 | 0 | 0 | 0 | 0 | 4 |
| 7 | AT1 | 0 | 0 | 0 | 0 | 0 | 0 | 9 |
| 8 | 101941B | 0 | 0 | 0 | 0 | 0 | 0 | 9 |
| 9 | 101850A | 0 | 0 | 0 | 0 | 0 | 0 | 7 |
| 10 | 101842C | 0 | 0 | 0 | 0 | 0 | 0 | 4 |
| 11 | PV63S | 0 | 0 | 0 | 0 | 0 | 7 | 9 |
| 12 | PF211 | 0 | 0 | 0 | 0 | 0 | 0 | 0 |
| 13 | Cn6966 | 0 | 0 | 0 | 0 | 0 | 0 | 9 |
| 14 | 102054I | 0 | 0 | 0 | 0 | 0 | 0 | 7 |
| 15 | PF287 | 0 | 0 | 0 | 0 | 0 | 0 | 9 |
| 16 | PrZ103640E | 0 | 0 | 0 | 0 | 0 | 0 | 0 |
| 17 | PrZ103602 | 0 | 0 | 0 | 0 | 0 | 0 | 9 |
| 18 | PF282 | 0 | 0 | 0 | 0 | 0 | 0 | 5 |
| 19 | Pf292 | 0 | 0 | 0 | 0 | 0 | 0 | 1 |
| 20 | PLF11R | 0 | 0 | 0 | 0 | 0 | 0 | 9 |

| | Dilution % | 0.16 | 0.08 | 0.04 | 0.02 | 0.01 | 0.005 |
|---|---|---|---|---|---|---|---|
| 1 | AT4 | 9 | 9 | 9 | 9 | 9 | 9 |
| 2 | P506 | 9 | 9 | 9 | 9 | 9 | 9 |
| 3 | P37 | 9 | 9 | 9 | 9 | 9 | 9 |
| 4 | NCTC 737 | 9 | 9 | 9 | 9 | 9 | 9 |
| 5 | PF284 | 9 | 9 | 9 | 9 | 9 | 9 |
| 6 | PF286 | 9 | 9 | 9 | 9 | 9 | 9 |
| 7 | AT1 | 9 | 9 | 9 | 9 | 9 | 9 |
| 8 | 101941B | 9 | 9 | 9 | 9 | 9 | 9 |
| 9 | 101850A | 9 | 9 | 9 | 9 | 9 | 9 |
| 10 | 101842C | 9 | 9 | 9 | 9 | 9 | 9 |
| 11 | PV63S | 9 | 9 | 9 | 9 | 9 | 9 |
| 12 | PF211 | 9 | 9 | 9 | 9 | 9 | 9 |
| 13 | Cn6966 | 9 | 9 | 9 | 9 | 9 | 9 |
| 14 | 102054I | 9 | 9 | 9 | 9 | 9 | 9 |
| 15 | PF287 | 9 | 9 | 9 | 9 | 9 | 9 |
| 16 | PrZ103640E | 9 | 9 | 9 | 9 | 9 | 9 |
| 17 | PrZ103602 | 9 | 9 | 9 | 9 | 9 | 9 |
| 18 | PF282 | 9 | 9 | 9 | 9 | 9 | 9 |
| 19 | Pf292 | 9 | 9 | 9 | 9 | 9 | 9 |
| 20 | PLF11R | 9 | 9 | 9 | 9 | 9 | 9 |

Strains showing antibiotic resistance are identified in bold type.

TABLE 8

| | Organism ID | MIC (mg/L) |
|---|---|---|
| 1 | AT4 | 0.31 |
| 2 | P506 | 0.31 |
| 3 | P37 | 0.16 |
| 4 | NCTC 737 | 0.31 |
| 5 | PF284¹ | 0.31 |
| 6 | PF286 | 0.31 |
| 7 | AT1 | 0.31 |
| 8 | 101941B | 0.31 |
| 9 | 101850A | 0.31 |
| 10 | 101842C | 0.31 |
| 11 | PV63S | 0.63 |
| 12 | PF211 | 0.16 |
| 13 | Cn6966 | 0.31 |
| 14 | 102054I | 0.31 |
| 15 | PF287 | 0.31 |
| 16 | PrZ103640E | 0.16 |
| 17 | PrZ103602 | 0.31 |
| 18 | PF282 | 0.31 |
| 19 | Pf292 | 0.16 |
| 20 | PLF11R | 0.31 |

| | |
|---|---|
| [MIC90] | 0.31 |

Strains showing antibiotic resistance are identified in bold type.

### Example 10

### Inhibition of Propinibacterium Acnes by Compositions of the Invention

The inhibition of Propionibacterium acnes by a composition of the invention in the form of a spot gel as defined in Example 4 was examined according to the general procedure identified in Example 8. Results of inhibition testing are shown in Table 9 and calculated MIC values are shown in Table 10.

TABLE 9

| | Dilution % | 10 | 5.0 | 2.5 | 1.25 | 0.63 | 0.31 | 0.16 |
|---|---|---|---|---|---|---|---|---|
| 1 | AT4 | 0 | 0 | 0 | 0 | 0 | 0 | 9 |
| 2 | P506 | 0 | 0 | 0 | 0 | 0 | 0 | 9 |
| 3 | P37 | 0 | 0 | 0 | 0 | 0 | 0 | 3 |
| 4 | NCTC 737 | 0 | 0 | 0 | 0 | 0 | 0 | 9 |
| 5 | PF284¹ | 0 | 0 | 0 | 0 | 0 | 0 | 9 |
| 6 | PF286 | 0 | 0 | 0 | 0 | 0 | 0 | 9 |
| 7 | AT1 | 0 | 0 | 0 | 0 | 0 | 0 | 9 |
| 8 | 101941B | 0 | 0 | 0 | 0 | 0 | 0 | 9 |
| 9 | 101850A | 0 | 0 | 0 | 0 | 0 | 0 | 9 |
| 10 | 101842C | 0 | 0 | 0 | 0 | 0 | 0 | 9 |
| 11 | PV63S | 0 | 0 | 0 | 0 | 0 | 9 | 9 |
| 12 | PF211 | 0 | 0 | 0 | 0 | 0 | 0 | 4 |
| 13 | Cn6966 | 0 | 0 | 0 | 0 | 0 | 0 | 9 |
| 14 | 102054I | 0 | 0 | 0 | 0 | 0 | 0 | 9 |
| 15 | PF287 | 0 | 0 | 0 | 0 | 0 | 4 | 9 |
| 16 | PrZ103640E | 0 | 0 | 0 | 0 | 0 | 0 | 4 |
| 17 | PrZ103602 | 0 | 0 | 0 | 0 | 0 | 0 | 9 |
| 18 | PF282 | 0 | 0 | 0 | 0 | 0 | 0 | 9 |
| 19 | Pf292 | 0 | 0 | 0 | 0 | 0 | 0 | 9 |
| 20 | PLF11R | 0 | 0 | 0 | 0 | 0 | 0 | 9 |

| | Dilution % | 0.16 | 0.08 | 0.04 | 0.02 | 0.01 | 0.005 |
|---|---|---|---|---|---|---|---|
| 1 | AT4 | 9 | 9 | 9 | 9 | 9 | 9 |
| 2 | P506 | 9 | 9 | 9 | 9 | 9 | 9 |
| 3 | P37 | 9 | 9 | 9 | 9 | 9 | 9 |
| 4 | NCTC 737 | 9 | 9 | 9 | 9 | 9 | 9 |
| 5 | PF284 | 9 | 9 | 9 | 9 | 9 | 9 |
| 6 | PF286 | 9 | 9 | 9 | 9 | 9 | 9 |
| 7 | AT1 | 9 | 9 | 9 | 9 | 9 | 9 |
| 8 | 101941B | 9 | 9 | 9 | 9 | 9 | 9 |
| 9 | 101850A | 9 | 9 | 9 | 9 | 9 | 9 |
| 10 | 101842C | 9 | 9 | 9 | 9 | 9 | 9 |
| 11 | PV63S | 9 | 9 | 9 | 9 | 9 | 9 |
| 12 | PF211 | 9 | 9 | 9 | 9 | 9 | 9 |
| 13 | Cn6966 | 9 | 9 | 9 | 9 | 9 | 9 |
| 14 | 102054I | 9 | 9 | 9 | 9 | 9 | 9 |
| 15 | PF287 | 9 | 9 | 9 | 9 | 9 | 9 |
| 16 | PrZ103640E | 9 | 9 | 9 | 9 | 9 | 9 |
| 17 | PrZ103602 | 9 | 9 | 9 | 9 | 9 | 9 |
| 18 | PF282 | 9 | 9 | 9 | 9 | 9 | 9 |
| 19 | Pf292 | 9 | 9 | 9 | 9 | 9 | 9 |
| 20 | PLF11R | 9 | 9 | 9 | 9 | 9 | 9 |

Strains showing antibiotic resistance are identified in bold type.

TABLE 10

| | Organism ID | MIC (mg/L) |
|---|---|---|
| 1 | AT4 | 0.31 |
| 2 | P506 | 0.31 |
| 3 | P37 | 0.16 |
| 4 | NCTC 737 | 0.31 |
| 5 | PF284¹ | 0.31 |
| 6 | PF286 | 0.31 |
| 7 | AT1 | 0.31 |
| 8 | 101941B | 0.31 |
| 9 | 101850A | 0.31 |
| 10 | 101842C | 0.31 |
| 11 | PV63S | 0.63 |
| 12 | PF211 | 0.31 |
| 13 | Cn6966 | 0.31 |
| 14 | 102054I | 0.31 |
| 15 | PF287 | 0.63 |
| 16 | PrZ103640E | 0.31 |
| 17 | PrZ103602 | 0.31 |
| 18 | PF282 | 0.31 |
| 19 | Pf292 | 0.31 |
| 20 | PLF11R | 0.31 |

| | |
|---|---|
| [MIC90] | 0.31 |

Strains showing antibiotic resistance are identified in bold type.

The results in Examples 9 and 10 show that both cream and gel compositions according to the invention demonstrate antimicrobial activity against a 20-isolate Propionibacterium Spp. panel at concentrations up to 10% (w/v) of product, with 18/20 (90%) of isolates having an MIC of ≦0.31 (w/v) of product.

Both of the compositions tested are equally effective against antibiotic-sensitive and antibiotic-resistant strains of Propinibacterium Spp. These results indicate that both of the tested compositions would be suitable for treatment of acne.

1. A composition comprising: (i) chlorhexidine or a pharmaceutically acceptable salt thereof; (ii) niacinamide or niacin; and (iii) salicylic acid or a pharmaceutically acceptable salt or derivative thereof.

2-3. (canceled)

4. A composition according to claim 1 comprising chlorhexidine digluconate.

5. A composition according to claim 1, comprising niacinamide.

6. (canceled)

7. A composition according to claim 1, comprising salicylic acid.

8-18. (canceled)

19. A composition according to claim 1, further comprising eucalyptol.

20-22. (canceled)

23. A composition according to claim 1, further comprising zinc-PCA.

24. (canceled)

25. A composition according to claim 1, further comprising one or more antioxidants.

26-27. (canceled)

28. A composition according to claim 1, further comprising chamomile extract.

29. (canceled)

30. A composition according to claim 4, wherein the amount of chlorhexidine digluconate is from 0.05 to 0.3 wt %.

31. A composition according to claim 30, wherein the amount of niacinamide is from 0.5 to 5 wt %.

32. A composition according to claim 31, wherein the amount of salicylic acid is from 0.2 to 5 wt %.

33. A composition according to claim 32, wherein the salicylic acid is encapsulated and has a number average particle size of less than 1.0 μm.

34. (canceled)

35. A composition according to claim 32, further comprising eucalyptol in an amount of from 0.3 to 5 wt %.

36. (canceled)

37. A composition according to claim 32, further comprising zinc-PCA in an amount of from 0.1 to 3 wt %.

38. A composition according to claim 32, further comprising tocopherol in an amount of from 0.5 to 5 wt %.

39. A composition according to claim 32, further comprising Goji extract in an amount of from 0.5 to 5 wt %.

40. A composition according to claim 32, further comprising chamomile extract in an amount of from 0.5 to 3 wt %.

41. A composition according to claim 1, in the form of a pharmaceutical composition suitable for topical administration.

42-44. (canceled)

45. A composition according to claim 41, further comprising one or more excipients suitable for topical administration.

46. (canceled)

47. A textile cloth, wipe, pad or face mask impregnated with a composition as defined in claim 1.

48. A textile cloth, wipe, pad or face mask according to claim 47, wherein the composition is as defined in claim 32.

49-50. (canceled)

51. A method for treating and/or preventing a skin disorder associated with inflammation of the skin, comprising contacting the skin with a composition as defined in claim 1 in an amount effective to treat or prevent the skin disorder.

52. (canceled)

53. The method according to claim 51, wherein the skin disorder is selected from the group consisting of bacterial dermatoses, allergic dermatoses, allergic dermatoses, pruritic dermatoses, vascular dermatoses, viral dermatoses, mycolic skin infections, granulomatous skin infections, parasitic skin infections, pigmented dermatoses, rosacea and photosensitive dermatoses.

54. The method according to claim 53, wherein the skin disorder is bacterial dermatoses.

55. (canceled)

56. The method according to claim 54, wherein the bacterial dermatoses are selected from the group consisting of acne vulgaris, impetigo, eethyma, folliculitis, carbuncles, sweat gland infections, erysipelas, erythrasma, infected ulcers, infected eczematoid dermatoses, rosacea and bacterial dermatoses associated with systemic infections.

57-70. (canceled)
